(19) United States
(12) Patent Application Publication
(10) Pub. No.: US 2025/0361499 A1
(43) Pub. Date: Nov. 27, 2025

(54) Thrombolytic Protease Resistant ADAMTS13 Mutants

(71) Applicant: McMaster University, Hamilton (CA)

(72) Inventors: Colin Kretz, Mount Hope (CA); Hasam Madarati, Hannon (CA); Veronica DeYoung, Trent Lakes (CA)

(21) Appl. No.: 18/872,220

(22) PCT Filed: Jun. 6, 2023
(86) PCT No.: PCT/CA2023/050770

§ 371 Date: Dec. 5, 2024

Related U.S. Application Data
(60) Provisional application No. 63/349,242, filed on Jun. 6, 2022.

(51) Int. Cl. C12N 9/64; A61K 38/00; A61K 38/48; A61K 45/06

(52) U.S. Cl. CPC C12N 9/6489; A61K 38/4886; A61K 45/06; C12Y 304/24087; A61K 38/00

(57) ABSTRACT

A protease-resistant ADAMTS13 mutant protein or nucleic acid encoding the ADAMTS13 mutant protein is provided. The ADAMTS13 mutant protein comprises a mammalian ADAMTS13 protein in which one or more protease cleavage sites within the protein are replaced with amino acid sequence that is resistant to protease cleavage, and the mutant protein retains von Willebrand factor (VWF)-cleaving activity. The protease-resistant ADAMTS13 mutant is useful as a thrombolytic agent to treat common thrombotic disorders, including stroke, myocardial infarction, venous thromboembolism, and rare microvascular thrombotic disorders like thrombotic thrombocytopenia purpura (TTP).

### FIELD

The present application relates generally to cardiovascular disease and thrombosis, and more particularly to a novel thrombolytic therapeutic agent, methods of making and uses thereof.

### BACKGROUND

Thrombosis refers to the pathological formation of a blood clot and is responsible for 1 in 4 deaths worldwide. Blood clots are comprised of activated platelets and polymerized fibrin. Current thrombosis treatments include anticoagulants and anti-platelet therapies to prevent fibrin formation or platelet activation, respectively. Thrombolytic therapy can be used in some cases to remove blood clots; however, because these therapies are based on t-PA, they only degrade the fibrin component of a blood clot and are associated with a significant risk of bleeding due to non-specific cleavage of insoluble fibrin in clots and soluble fibrinogen in circulation, which limits their use. There is currently no available thrombolytic therapy that targets the platelet component of a blood clot.

Platelets are recruited to blood clots by the multimeric plasma protein Von Willebrand Factor (VWF)(1). VWF is secreted from endothelial cells as an ultra-large multimer that requires processing into a distribution of multimer lengths that provide a balanced blood clotting system. VWF has many roles in biology, including in thrombosis and inflammation and has been the target of several pharmacological agents to treat cardiovascular disease (12-14). ADAMTS13 is a protease that specifically degrades VWF localized to sites of injury where shear stresses permit proteolysis, and reduces its capacity to recruit platelets to blood clots(1). High VWF activity and low ADAMTS13 activity are associated with common thrombotic disorders, including stroke, myocardial infarction, and venous thromboembolism. ADAMTS13 can be downregulated by proteases present at sites of thrombosis and inflammation, potentially reducing its effectiveness at downregulating VWF. Importantly, unlike plasmin (the fibrinolytic protease generated by tPA-based therapies), ADAMTS13 only has one substrate, VWF.

ADAMTS13 is a 180 kDa plasma metalloprotease predominantly synthesized in the liver by hepatic stellate cells and secreted into circulation as an active enzyme (2). VWF multimers can be cleaved by ADAMTS13 during secretion from endothelial cells and at a developing blood clot site and is dependent on fluid shear stresses (1). The shear-dependency protects VWF from indiscriminate degradation by ADAMTS13 in circulation and limits the risk of bleeding.

Deficiency in ADAMTS13 is associated with common thrombotic disorders, including stroke, myocardial infarction, sepsis, and venous thromboembolism (3-6). ADAMTS13 deficiency is also associated with rare microvascular thrombotic disorders like thrombotic thrombocytopenia purpura (TTP). TTP is primarily caused by developing auto-inhibitory antibodies against ADAMTS13 but can also be caused by a genetic deficiency in ADAMTS13 in rare cases. This loss of ADAMTS13 levels (<5% activity) leads to unregulated VWF multimer lengths and the spontaneous deposition of VWF/platelet-rich aggregates in the microvasculature (3).

Recombinant ADAMTS13 is in clinical trials to treat TTP and other cardiovascular diseases. Recombinant ADAMTS13 has also been shown to improve outcomes in preclinical models of stroke and myocardial infarction (7, 8). Recombinant ADAMTS13 reduces inflammation, platelet recruitment, and microvascular thrombosis suggesting broad utility to treat chronic and acute cardiovascular disease (7-10).

ADAMTS13 is degraded by various proteases of the coagulation and fibrinolytic system and by proteases released by activated immune cells, such as neutrophils (11). These proteases are present at sites of thrombosis.

The background herein is included solely to explain the context of the disclosure. This is not to be taken as an admission that any of the material referred to was published, known, or part of the common general knowledge as of the priority date.

### SUMMARY

Mutant forms of ADAMTS13 have now been developed that are resistant to degradation by proteases generated at the sites of blood vessel injury, and are, therefore, effective antithrombotic and thrombolytic agents.

Thus, in one aspect of the invention, a protease-resistant ADAMTS13 mutant protein or nucleic acid encoding the ADAMTS13 mutant protein is provided, wherein the ADAMTS13 mutant protein comprises a mammalian ADAMTS13 protein in which one or more protease cleavage sites within the protein are replaced with amino acid sequence that is resistant to protease cleavage, and wherein the mutant protein retains VWF-cleaving activity.

In another aspect, a composition comprising a protease-resistant ADAMTS13 mutant is provided.

In a further aspect, a method of inhibiting or at least reducing thrombosis is provided comprising administering to a mammal in need a protease-resistant ADAMTS13 mutant or nucleic acid encoding the ADAMTS13 mutant protein as herein described.

The present application's other features and advantages will become apparent from the following detailed description. It should be understood, however, that the detailed description and the specific examples, while indicating embodiments of the application, are given by way of illustration only, and the scope of the claims should not be limited by these embodiments but should be given the broadest interpretation consistent with the description as a whole.

### DRAWINGS

The embodiments of the application will now be described in greater detail concerning the attached drawings in which:

FIG. 1 shows the degradation sites of various proteases on ADAMTS13.

FIG. 2 shows the ADAMTS13 domains, the amino acid sequence at T4L and T8L and their mutations.

FIG. 3 shows the T4L, T8L, and T4L/T8L are resistant to proteolysis by plasmin, hPR3, Cathepsin G, FXIa, thrombin, kallikrein, and elastase.

FIG. 4 illustrates graphically the results of a plasma thrombin generation assay (A) and ADAMTS13 degradation by thrombin was visualized via Western Blot (B) and shows T4L/T8L resistance to degradation by coagulation factor plasma stimulated with recombiplastin.

FIG. 5 illustrates graphically the results of ADAMTS13 and T4L/T8L mutant degradation studies in a plasma fibrinolysis assay (A) and cleavage was visualized via Western Blot (B) which shows T4L/T8L resistance to fibrinolytic proteases in plasma stimulated with recombiplastin and tPA.

FIG. 6 shows T4L/T8L is comparable to wild type ADAMTS13 for cleaving FRET-VWF73.

FIG. 7 shows T4L/T8L is comparable to wild type ADAMTS13 for cleaving VWF/platelet complexes on endothelial cells under flow.

FIG. 8 shows the T4L/T8L/1380G mutant resistance to proteolysis by neutrophil elastase.

FIG. 9 shows the T4L/T8L/1380G mutant, T4L/T8L mutant, and wild type ADAMTS13 resistance to proteolysis by activated neutrophils.

FIG. 10 illustrates the A) amino acid and B) mRNA transcript sequence of human ADAMTS13.

FIG. 11 illustrates: A) wild-type amino acid sequence of ADAMTS13; B) amino acid sequence for T4L mutant ADAMTS13; C) amino acid sequence for T8L mutant ADAMTS13; D) amino acid sequence of T4L/T8L double linker mutant; and E) amino acid sequence for T4L/T8L/1380G mutant ADAMTS13.

### DETAILED DESCRIPTION

A protease-resistant ADAMTS13 mutant protein or nucleic acid encoding the ADAMTS13 mutant protein is provided. The ADAMTS13 mutant protein comprises a mammalian ADAMTS13 protein in which one or more protease cleavage sites within the protein are replaced with amino acid sequence that is resistant to protease cleavage, and the mutant protein retains VWF-cleaving activity.

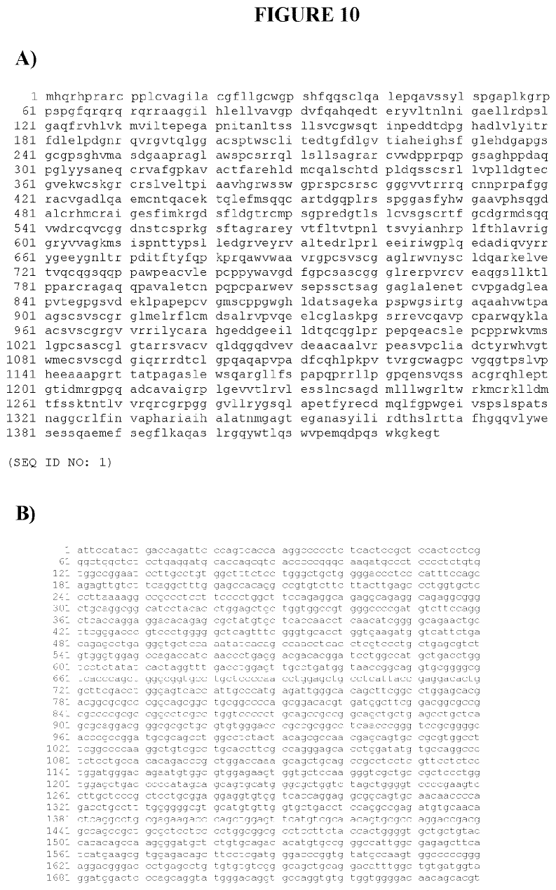

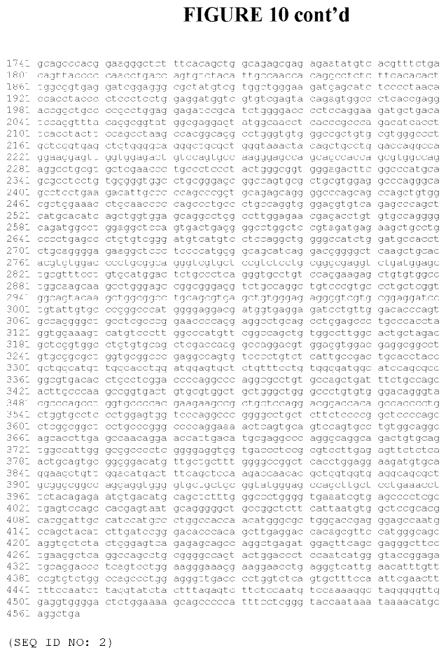

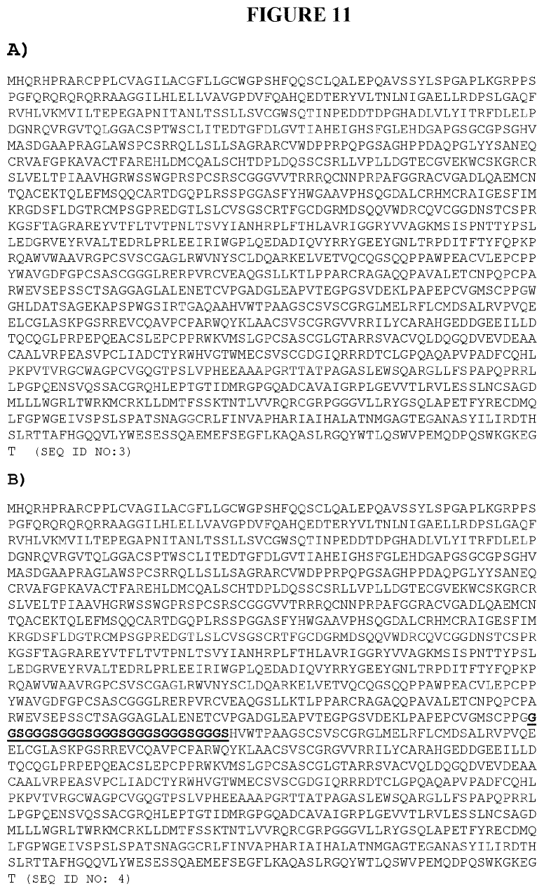

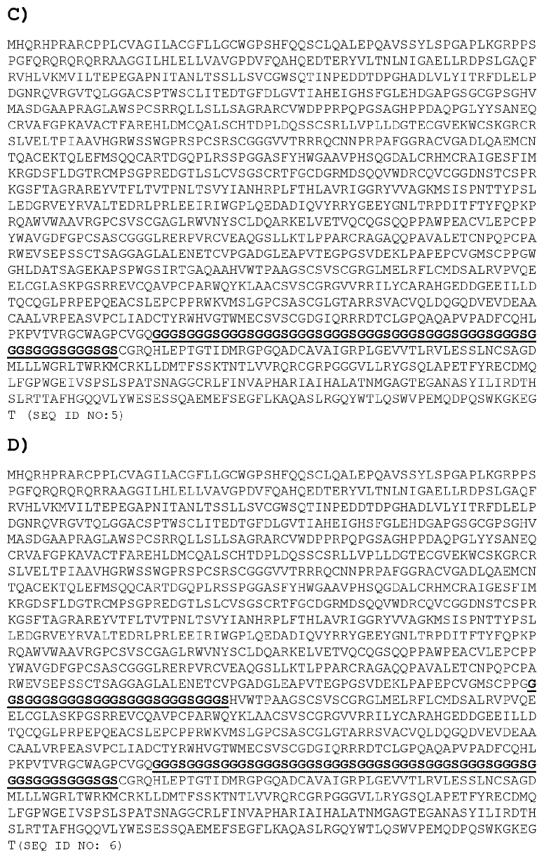

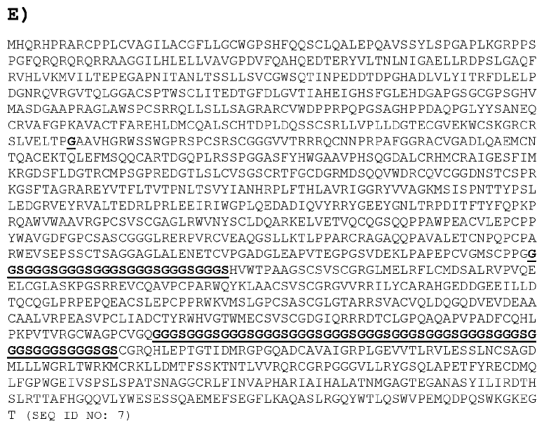

The term “ADAMTS13” or “A Disintegrin and Metalloproteinase with a Thrombospondin type 1 motif, member 13”, also known as von Willebrand factor-cleaving protease (VWFCP), is a zinc-containing metalloprotease enzyme that cleaves VWF. It is secreted into the blood and degrades large VWF multimers, decreasing their activity. ADAMTS13 refers to the mammalian protein, and functionally equivalent isoforms and variants thereof, including ADAMTS13 from other species. The human ADAMTS13 protein sequence and mRNA transcript encoding it are provided in FIG. 10.

The term “functionally equivalent” with respect variants (e.g. isoforms) of ADAMTS13 refers to forms of ADAMTS13 that retain VWF cleaving activity. The term “functionally equivalent” encompasses both naturally and non-naturally occurring variants of ADAMTS13 that retain the biological activity of ADAMTS13, e.g. to cleave VWF. The variant need not exhibit identical activity to endogenous ADAMTS13, but will exhibit sufficient activity to render it useful to cleave VWF, e.g. at least about 25% of the biological activity of native ADAMTS13, and preferably at least about 50% or greater of the biological activity of ADAMTS13. In embodiments, variants of ADAMTS13 may possess greater activity than the native version thereof. Such functionally equivalent variants may result naturally from alternative splicing during transcription or from genetic coding differences and may retain significant sequence homology with wild-type ADAMTS13, e.g. at least about 70% sequence homology, preferably at least about 80% sequence homology, and more preferably at least about 90% or greater sequence homology. Such variants can readily be identified using established cloning techniques employing primers derived from ADAMTS13. Additionally, such modifications may result from non-naturally occurring synthetic alterations made to ADAMTS13 to render functionally equivalent variants which may have more desirable characteristics for use in a therapeutic sense, for example, increased activity or stability. Non-naturally occurring variants of ADAMTS13 include analogues, fragments and derivatives thereof.

A functionally equivalent analogue of ADAMTS13 in accordance with the present invention may incorporate one or more amino acid substitutions, including additions and/or deletions. Amino acid additions or deletions include both terminal and internal additions or deletions to yield a functionally equivalent peptide. Examples of suitable amino acid substitutions include those made at positions within the protein that are not closely linked to activity, for example, the type 1 thrombospondin repeats 1-8, as well as conservative amino acid substitutions since such substitutions are less likely to adversely affect function. Examples of conservative substitutions include the substitution of a non-polar (hydrophobic) residue such as alanine, isoleucine, valine, leucine or methionine with another non-polar (hydrophobic) residue; the substitution of a polar (hydrophilic) residue with another such as between arginine and lysine, between glutamine and asparagine, between glutamine and glutamic acid, between asparagine and aspartic acid, and between glycine and serine; the substitution of a basic residue such as lysine, arginine or histidine with another basic residue; or the substitution of an acidic residue, such as aspartic acid or glutamic acid with another acidic residue.

A functionally equivalent fragment in accordance with the present invention comprises a portion of ADAMTS13 sequence which maintains the VWF cleaving function of intact ADAMTS13, such as N-or C-terminally truncated fragments. Such biologically active fragments of ADAMTS13 can readily be identified using assays useful to evaluate the VWF cleaving activity of ADAMTS13 protein such as those herein described.

A functionally equivalent derivative of ADAMTS13 in accordance with the present invention is ADAMTS13, or an analogue or fragment thereof, in which one or more of the amino acid residues therein is chemically derivatized. The amino acids may be derivatized at the amino or carboxy groups, or alternatively, at the side “R” groups thereof. Derivatization of amino acids within the peptide may yield a peptide having more desirable characteristics for use as a therapeutic such as increased stability or enhanced activity. Such derivatized molecules include for example, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form, for example, salts, methyl and ethyl esters or other types of esters or hydrazides. Free hydroxyl groups may be derivatized to form, for example, O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids, for example: 4-hydroxyproline may be substituted for proline; 5-hydroxylysine may be substituted for lysine; 3-methylhistidine may be substituted for histidine; homoserine may be substituted for serine; and ornithine may be substituted for lysine. Terminal derivatization of the protein to protect against chemical or enzymatic degradation is also encompassed including acetylation at the N-terminus and amidation at the C-terminus of the peptide.

ADAMTS13, and functionally equivalent variants thereof, may be made using standard, well-established solid-phase peptide synthesis methods (SPPS). Two methods of solid phase peptide synthesis include the BOC and FMOC methods. ADAMTS13nand variants thereof may also be made using any one of a number of suitable techniques based on recombinant technology. It will be appreciated that such techniques are well-established by those skilled in the art, and involve the expression of ADAMTS13-encoding nucleic acid in a genetically engineered host cell. Nucleic acid encoding ADAMTS13 may be synthesized de novo by automated techniques also well-known in the art given that the protein and nucleic acid sequences are known.

ADAMTS13-encoding nucleic acid molecules or oligonucleotides may also be used to increase plasma ADAMTS13 levels in a mammal. In this regard, “ADAMTS13-encoding nucleic acid” is used herein to encompass mammalian ADAMTS13-encoding nucleic acid, including human and non-human forms, and functionally equivalent forms thereof (e.g. that encode functionally equivalent ADAMTS13, or nucleic acids which differ therefrom due to degeneracy of the genetic code).

The term “oligonucleotide” refers to an oligomer or polymer of nucleotide or nucleoside monomers consisting of naturally occurring bases, sugars, and intersugar (backbone) linkages. The term also includes modified or substituted oligonucleotides comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide. Other oligonucleotides of the invention may contain modified phosphorous, oxygen heteroatoms in the phosphate backbone, short chain alkyl or cycloalkyl intersugar linages or short chain heteroatomic or heterocyclic intersugar linkages. For example, oligonucleotides may contain phosphorothioates, phosphotriesters, methyl phosphonates, and phosphorodithioates. Oligonucleotides of the invention may also comprise nucleotide analogs such as peptide nucleic acid (PNA) in which the deoxribose (or ribose) phosphate backbone in the DNA (or RNA), is replaced with a polyamide backbone similar to that found in peptides. Other oligonucleotide analogues may contain nucleotides containing polymer backbones, cyclic backbones, or acyclic backbones, e.g. morpholino backbone structures.

Such oligonucleotide molecules are readily synthesized using procedures known in the art based on the available sequence information. For example, oligonucleotides may be chemically synthesized using naturally occurring nucleotides or modified nucleotides as described above designed to increase the biological stability of the molecules or to increase the physical stability of the duplex formed with mRNA or the native gene, e.g. phosphorothioate derivatives and acridine substituted nucleotides. Selected oligonucleotides may also be produced biologically using recombinant technology in which an expression vector, e.g. plasmid, phagemid or attenuated virus, is introduced into cells in which the oligonucleotide is produced under the control of a regulatory region.

According to an aspect of the present invention, a mutant of ADAMTS13, or a functionally equivalent variant thereof, is provided which is protease resistant, i.e. resistant to cleavage by proteases. In particular, the ADAMTS13 protein is mutated within protease recognition/cleavage sites. The mutations are sufficient to prevent or reduce protease cleavage of the ADAMTS13 protein at the mutated site while not having an adverse effect on ADAMTS13 activity, namely VWF cleaving activity. In this regard, it is noted that while it is desirable to essentially fully retain endogenous VWF activity within an ADAMTS13 mutant, mutants which at least partially retain VWF cleaving activity are also considered functional for the purposes of the invention. In one embodiment, ADAMTS13 protein is mutated within a cleavage site of a protease involved in coagulation or fibrinolytic activity, or within a cleavage side of a protease released by activated neutrophils, but retains VWF cleaving activity. In another embodiment, the ADAMTS13 protein is mutated within a cleavage site which is sensitive to cleavage by a serine protease, for example, a cleavage site which is sensitive to cleavage by at least thrombin, and which may additionally also be sensitive to cleavage by one or more of enzymes selected from plasmin, FXa, FXIa, kallikrein, cathepsin G, elastase, and HPR3. In another embodiment, ADAMTS13 is mutated within one or more linking regions in the ADAMTS13 sequence, such as a linking region within the TSP repeat region (between the 4^{th} and 5^{th} TSP-T4L), e.g. W848-A894, and/or a linking region between the TSP and CUB domain (T8L), e.g. G1134-A119. In other embodiments, the ADAMTS13 mutant comprises the sequence GGS [GGGS]₆ at amino acid position 848-894 and/or the sequence [GGGS]₁₄GS at position 1134-1191.

In a further embodiment, ADAMTS13 is mutated to become neutrophil elastase insensitive. The cleavage site for neutrophil elastase is within the disintegrin-like domain (Dis) of ADAMTS13, which spans, for example, the amino acid residues at positions 287-383 of the human protein. In an embodiment, the Dis neutrophil elastase cleavage site is mutated to become resistant to cleavage by neutrophil elastase. In another embodiment, the amino acid at position 380 is mutated to become resistant to elastase cleavage. In another embodiment, the elastase cleavage site is mutated to replace an isoleucine with a glycine residue (I380G).

The ADAMTS13 protein is mutated to replace a protease cleavage site with a sequence that is protease resistant, or not protease-sensitive, i.e. which is not cleaved by a protease, such as a protease involved in coagulation/fibrinolysis or released by activated immune cells, and which does not otherwise adversely affect the activity of the ADAMTS13 protein. In one embodiment, the cleavage site is replaced with an amino acid sequence which is resistant to protease cleavage, for example, a glycine-rich linker sequence, such as a glycine-serine linker sequence. Suitable glycine-rich linker sequences comprise from about 20-100 amino acid residues, with a glycine content of at least about 25%, preferably at least 50% or greater glycine content. Exemplary linkers may comprise a GGS or GGGS repeat, e.g. 3 to 25 repeats, and may include additional GS or GGS residues at either end thereof. The linker may correspond in size with the region to be replaced, or may be larger or smaller than the region being replaced, as long as the mutated protein retains VWF function. As set out above, ADAMTS13 mutants may be prepared using chemical or biological methods known in the art. Alternatively, the mutants may be prepared using gene editing techniques to edit ADAMTS13-encoding nucleic acid to encode a selected mutant. Gene editing techniques that may be used include, but are not limited to, methods using CRISPR technology, zinc-finger nucleases (ZFN), transcription activator-like effector nucleases (TALENs) and meganucleases.

Once prepared and suitably purified, ADAMTS13 mutant protein, ADAMTS13 mutant-encoding oligonucleotides, or functionally equivalent variants thereof, may be utilized in accordance with the invention to treat pathological conditions involving thrombosis. Examples of thrombotic disorders for which the present ADAMTS13 mutant is useful to treat, include, but are not limited to, acute and chronic cardiac conditions such as myocardial infarction and stroke, atherosclerosis, venous thromboembolism, pulmonary embolism and microvascular thrombotic disorders such as thrombotic thrombocytopeniaurpura and vaso-occlusive crisis such as that experienced by patients with sickle-cell disease. The present method is also useful to treat sepsis, colitis and diabetes. The term “treat” or “treatment” as used herein refers to the curing, reducing or preventing thrombosis, including but not limited to reducing one or more of the symptoms of thrombosis, including but not limited to inflammation and pain in an affected area (e.g. legs).

ADAMTS13 mutant or nucleic acid encoding ADAMTS13 mutant may be administered either alone or in combination with at least one pharmaceutically acceptable adjuvant, for use in treatments in accordance with embodiments of the invention. The expression “pharmaceutically acceptable” means acceptable for use in the pharmaceutical and veterinary arts, i.e. not being unacceptably toxic or otherwise unsuitable. Examples of pharmaceutically acceptable adjuvants are those used conventionally with peptide-or nucleic acid-based drugs, such as diluents, excipients and the like. Reference may be made to “Remington's: The Science and Practice of Pharmacy”, 21st Ed., Lippincott Williams & Wilkins, 2005, for guidance on drug formulations generally.

The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragacanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Formulations for administration intranasally, or by inhalation, may also be prepared in saline or other suitable buffer and/or propellant adjuvants, to be nebulized to form a liquid aerosol for inhalation by mouth or nasally. Other adjuvants may also be added to the composition regardless of how it is to be administered, for example, anti-microbial agents may be added to the composition to prevent microbial growth over prolonged storage periods.

Therapeutic ADAMTS13 mutant-encoding oligonucleotides may be directly administered in vivo formulated, for example, in saline or an appropriate buffer. Alternatively, the oligonucleotides may be introduced into tissues or cells ex vivo using techniques in the art including vectors (retroviral vectors, adenoviral vectors and DNA virus vectors) or by physical techniques such as microinjection, and then administered in vivo. Administration of such cells may be achieved, for example, by encapsulated cell biodelivery. DNA may also be delivered conjugated to nanoparticles, e.g. gold, silver, platinum or polymeric nanoparticles, or encapsulated within nanoparticles, e.g. encapsulated within a suitable polymer such as poly (DL-lactide-co-glycolide) polymer.

For use to treat or prevent thrombosis, a therapeutically effective amount of ADAMTS13 mutant or nucleic acid encoding ADAMTS13 mutant is administered to a mammal. As used herein, the term “mammal” is meant to encompass, without limitation, humans, domestic animals such as dogs, cats, horses, cattle, swine, sheep, goats and the like, as well as non-domesticated animals. The term “therapeutically effective amount” is an amount of the ADAMTS13 mutant or nucleic acid encoding ADAMTS13 mutant required to treat thrombosis, while not exceeding an amount which may cause significant adverse effects. The ADAMTS13 mutant functions by providing a more balanced non-pathogenic hemostatic ADAMTS13/VWF axis, e.g. by reducing the length of VWF multimers in circulation and at the site of blood vessel injury, and by reducing the accumulation of platelets and other immune cells at the site of blood vessel injury, so as to prevent or at least minimize the occurrence of thrombosis.

Dosages of ADAMTS13 mutant, functionally equivalent variants thereof, or nucleic acid encoding it, that are therapeutically effective will vary on many factors including the nature of the condition to be treated as well as the particular individual being treated. In embodiments, dosages of ADAMTS13 mutant in the range of about 0.1-500 mg, for example 1-10 mg, or 0.1-10 mg, or a dosage of nucleic acid encoding ADAMTS13 mutant that expresses about 0.1-500 mg of ADAMTS13 mutant, such as 1-10 mg of ADAMTS13 mutant. The dosage may be a single dosage, a total dosage administered over a period of time such as 2 or more days, or a daily dosage administered over a period of time, e.g. 2 or more days. As one of skill in the art will appreciate, pediatric dosages will generally be at the lower end of the recited range.

In the present treatment, ADAMTS13 mutant or mutant-encoding nucleic acid may be administered by any route suitable to increase the plasma levels thereof. Examples of suitable administrable routes include, but are not limited to, oral, subcutaneous, intravenous, intraperitoneal, intranasal, enteral, topical, sublingual, intramuscular, intra-arterial, intramedullary, intrathecal, inhalation, ocular, transdermal, vaginal or rectal means. Depending on the route of administration, the protein or nucleic acid may be coated or encased in a protective material to prevent undesirable degradation thereof by enzymes, acids or by other conditions that may affect the therapeutic activity thereof.

In one embodiment, ADAMTS13 mutant or nucleic acid encoding it may be administered alone, or in conjunction with (either combined together, or at the same time, simultaneously with or administered at different times) at least one other therapeutic compound such as another compound effective to treat or prevent thrombosis, including anticoagulants such as heparin, low molecular weight heparin (LMWH), antithrombin, antithrombin-heparin complexes and fondaparinux; direct oral anticoagulants (DOACs),such as rivaroxaban, apixaban, betrixaban, edoxaban, dabigatran, hirudin, bivalirudin, argatroban, thrombomodulin, corn-trypsin inhibitor and vitamin K antagonists; fibrinolytic agents such as tissue plasminogen activator (t-PA) and derivatives thereof such as, but not limited to Alteplase, Reteplase and Tenecteplase and urokinase plasminogen activator (u-PA); antifibrinolytic agents like tranexamic acid or epsilon-aminocaproic acid; and antiplatelet agents such as aspirin, adenosine diphosphate (ADP) receptor inhibitors, e.g. clopidogrel, ticagrelor and prasgrel, GPIIb/IIIa inhibitors, e.g. tirofiban, abciximab and eptifibatide; dipyridamole, prostacyclin and apyrase.

The present ADAMTS13 mutant or nucleic acid encoding may also be administered in conjunction with a therapeutic agent to treat one or more symptoms of thrombosis, such as inflammation and/or pain, e.g. a non-steroidal anti-inflammatory drug (NSAID) such as ibuprofen, naproxen, diclofenac, indomethacin, etoricoxib, mefanamic acid and celcoxib; or a non-NSAID such as acetaminophen. Other analgesic agents include opioids such as codeine, fentanyl, hydrocodone, meperidine, methadone, naloxone and oxycodone.

### Definitions

Unless otherwise indicated, the definitions and embodiments described in this and other sections are intended to apply to all embodiments and aspects of the present application herein described. They are suitable as would be understood by a person skilled in the art. It is also to be understood that the terminology used herein is for the purpose of describing particular aspects only and is not intended to be limiting.

The term T4L as used herein, refers to the protein ADAMTS13 mutated at W848-A894 with a glycine-serine hinge sequence GGS [GGGS] ₆.

The term T8L as used herein, refers to the protein ADAMTS13 mutated at G1134-A1191 with a glycine-serine hinge sequence [GGGS]₁₄GS.

The term T4L/T8L, as used herein, refers to the protein ADAMTS13 mutated at W848-A894 and G1134-A1191 with a glycine-serine hinge sequence GGS [GGGS]₆ and [GGGS]₁₄GS respectively.

The term T4L/T8L/1380G, as used herein, refers to the protein ADAMTS13 mutated at W848-A894 and G1134-A1191 with a glycine-serine hinge sequence GGS [GGGS]₆ and [GGGS]₁₄GS respectively, and at isoleucine 380 with a glycine residue.

It will be understood that any component defined herein as being included may be explicitly excluded by way of proviso or negative limitation, such as any specific compounds or method steps, whether implicitly or explicitly defined herein.

In understanding the scope of the present application, the term “comprising” and its derivatives, as used herein, are intended to be open-ended terms that specify the presence of the stated features, elements, components, groups, integers, and steps, but do not exclude the presence of other unstated features, elements, components, groups, integers and steps. The foregoing also applies to words having similar meanings, such as the terms “including,” “having,” and their derivatives. The term “consisting” and its derivatives, as used herein, is intended to be closed terms that specify the presence of the stated features, elements, components, groups, integers, and steps but exclude the presence of other unstated features, elements, components, groups, integers and steps. The term “consisting essentially of,” as used herein, is intended to specify the presence of the stated features, elements, components, groups, integers, and steps as well as those that do not materially affect the basic and novel characteristic(s) of features, elements, components, groups, integers, and steps.

Terms of degree such as “substantially,” “about,” and “approximately” as used herein mean a reasonable amount of deviation of the modified term such that the result is not significantly changed. These terms of degree should be construed as including a deviation of at least ±10% of the modified term if this deviation would not negate the meaning of the word it modifies.

As used in this disclosure, the singular forms “a,” “an,” and “the” include plural references unless the content dictates otherwise.

In embodiments comprising an “additional” or “second” component, the second component as used herein is chemically different from the other components or first component. A “third” component is different from the other first and second components, and further enumerated, or “additional” components are similarly different.

The term “and/or” as used herein means that the listed items are present, or used, individually or in combination. In effect, this term means that “at least one of” or “one or more” of the listed items is used or present.

The abbreviation “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

EXAMPLES

The following non-limiting examples are illustrative of the present application:

### Example 1

### Methods and Materials

The literature was searched for evidence of the proteolytic degradation of ADAMTS13 by various enzymes. In-silico approaches, such as PROSPER (Protease specificity prediction server—Monash University), ExPASy Peptide Cutter (SIB Swiss Institute of Bioinformatics), and NEBcutter (v2.0, New England Biolabs), predicted various neutrophil-derived enzymes, such as cathepsin G, cathepsin K, and elastase, and coagulation proteases, such as FXa, and thrombin, to cleave ADAMTS13 throughout various sites on ADAMTS13. Experimental findings utilized gel electrophoresis. They identified the relative proteolytic areas of the neutrophil-derived enzyme, elastase, cathepsin G, human proteinase 3, and coagulation enzymes, plasmin, thrombin, and FXIa onto ADAMTS13.

In-vitro proteolysis of ADAMTS13—Reactions were performed in a 50 uL reaction volumes containing 100 nM rhADAMTS13 (R&D Systems: 6156-AD-020) or purified full length-ADAMTS13 and 50 nM various recombinant protease in ADAMTS13 reaction buffer (20 mM Tris-HCl, pH 7.4, 150 mM NaCl, 5 mM CaCl2, 10uM ZnCl2, 0.01% Tween 20). These proteases include coagulation proteases purchased from Haematologic Technologies: thrombin, fXa, flXa, fXIa, fXIIa, kallikrein, fVIIa, plasmin, uPa, tPa, and neutrophil derived proteases purchased from Sigma including: neutrophil elastase, cathepsin G, and proteinase 3. ADAMTS13 was incubated for varying time points (0-3 h) with various recombinant proteases, at 37° C. Reactions were were stopped using SDS-loading dye and separated via SDS-PAGE under reducing conditions. SDS-PAGE gels were either stained using SYPRO-RUBY for total protein analysis or by western blot using polyclonal anti-ADAMTS13 antibody (Abcam: ab28274) and goat anti-rabbit HRP-conjugated antibody (Bir-Rad: 1706515).

Isolation of Neutrophils—10 mL of blood was collected in a 20 mL syringe containing 2.5 mL citrate at room temperature. Then, 2.5 mL of dextran (6% dextran in saline) was drawn up by BD Vacutainer into the citrated blood and mixed slowly 4 times in a FIG. 8 movement. The leukocyte-rich plasma layer was left to separate by gravity for 1 hour. Histopaque 1077 (Sigma: 10771), at room temperate, and at an equal volume to that of separated leukocyte-rich plasma layer (˜6-8 mL), was added to a 50 mL tube. The separated leukocyte-rich plasma layer was added slowly to a new 50 mL tube. Using a serological pipette, the plasma layer was transferred and added onto the superficial layer of the Histopaque solution at the slowest rate possible. The plasma-Histopaque mixture was centrifuged at 1200×g, for 20 minutes with an acceleration of 7 and deceleration of 0. The supernatant was aspirated, and 2.5 mL of ACK lysis buffer was added onto the pellet. The pellet was slowly resuspended and incubated in the buffer for 4 minutes. Hank's Balanced Salt Solution (HBSS, GIBCO: 14065-056) was added to 50 mL then centrifuged at 1200 rpm, for 5minutes, at 4 degrees, with an acceleration and deceleration of 9. After that, the supernatant was discarded and if the pellet was red, the ACK lysis buffer step was repeated. The white pellet, i.e. the neutrophils, was resuspended slowly in 10 mL of RPMI (GIBCO: 11835-030). The cells were counted using trypan blue-PBS solution (GIBCO: 15250-061) and a hemocytometer. The volume corresponding to the number of cells needed in the corresponding experiment was transferred into a new 2- or 15-mL tube, centrifuged at 1200 rpm for 5 minutes, the supernatant was discarded, and the pelleted cells were resuspended in RPMI to the volume required to the corresponding experiment.

Neutrophil-containing reactions were set up in 40 L volume containing a varying number of neutrophils, 0-500×10³ cells diluted in RPMI, and 100 nM PMA for activation for 4 hours at 37° C. Neutrophils were activated in the presence or absence of 20 g/mL DNase 1. After that, 100 nM of recombinant or purified ADAMTS13 in ADAMTS13 kinetic buffer was added to the activated neutrophils, to a total volume of 50 μL. The mixture was incubated for 1 hour at 37° C., then analyzed by Western blot as described above. In experiments where the number of neutrophils was constant, 50,000 neutrophils were utilized, and the experiments were run. In the proteolysis experiments whereby specific inhibitors were utilized, Sivelstat (elastase inhibitor, Tocris: 3535) and Cathepsin G Inhibitor I (Millipore Sigma: 219372) were utilized and the experiments were run.

We took an in-silico approach to help identify sites on ADAMTS13 that are sensitive to cleavage by coagulation and immune proteases. Online tools capable of predicting cleavage sites proteolytically degraded by specific proteases in a specified peptide sequence, such as PROSPER (Protease specificity prediction server-Monash University), ExPASy Peptide Cutter (SIB Swiss Institute of Bioinformatics), and NEBcutter (v2.0, New England Biolabs). These predicted sites were prioritized based on results of the above in vitro studies in order to identify experimentally validated cleavage sites for the above listed proteases.

Our formal survey to find which proteases can cleave ADAMTS13 identified plasmin, thrombin, FXIa, FXa, tPa, kallikrein, cathepsin G, elastase, and hPR3 as enzymes capable of cleaving ADAMTS13.

ADAMTS13 mutants were designed using SeqBuilder 14, whereby the T4L, or T8L, or both, regions of ADAMTS13 were mutated to a variable length of GGGS repeats. The T4L mutant represents the mutation GGS [GGGS]₆ at W868-A894, and the T8L mutant represents the mutation [GGGS]₁₄GS at G1134-A1191. The T4L/T8L mutant represents both mutations. According to their mutated regions, these constructs were termed T4L, T8L, or T4L/T8L mutants. The T4L, T8L, and T4L/T8L mutants were genetically synthesized into pcDNA 3.1(+) from Bio Basic Inc. (Markham, ON, Canada). The 1380G mutation was made be performing site-directed mutagenesis on the T4L/T8L mutant. DNA vectors corresponding to each mutant, along with wt-ADAMTS13 in pcDNA 3.1(+), were transfected and expressed into HEK 293T cells. Expressed proteins in FreeStyle media were concentrated using centrifugal filters (Satorius Vivaspin 6-30,000 MWCO-VS0622) and quantified using the ELISA kit (R&D Systems: DADT130).

Proteolytic resistance by ADAMTS13 mutants: In-vitro proteolysis reactions of ADAMTS13 occurred using full length-ADAMTS13 (R&D Systems: 6156-AD-020), T4L-ADAMTS13, T8L-ADAMTS13, T4L/T8L-ADAMTS13, T4L/T8L/1380G-ADAMTS13. ADAMTS13 and each mutant were incubated with various recombinant proteases at 37° C. for 1-3 hours at the volume of 50 μL. Reactions were stopped using SDS-loading dye and separated via SDS-PAGE under reducing conditions. SDS-PAGE gels were analyzed through a western blot using a polyclonal anti-ADAMTS13 antibody (Abcam: ab28274) and goat anti-rabbit HRP-conjugated antibody (Bio-Rad: 1706515). Our findings on using a protease-resistant ADAMTS13 identified T4L/T8L-ADAMTS13 as resistant to proteolysis by plasmin, thrombin, FXIa, kallikrein, hPR3, Cathepsin, G, and elastase. The T4L/T8L/1380G ADAMTS13 was found to be resistant to neutrophil elastase.

Proteolytic resistance of T4L-ADAMTS13, T8L-ADAMTS13, T4L/T8L-ADAMTS13 to coagulation proteases in plasma: In a black, round-bottom, 96-well plate, 3.33 μL of phosphatidylcholine-phosphatidylserine lipid vesicles (PCPS) were added to give a final concentration of 15 μM, followed by 36.6 μL of human platelet-poor plasma, and 10 μL of undiluted, 1:10, 1:100, or 1:1000, or 0 tissue factor (HemosIL RecombiPlasTin: 00020301400). The reaction was incubated at 37° C. for 15 minutes. To initiate thrombin generation, 50 μL of substrate mixture (52 mM CaCl₂, 40 mM HEPES, 2 mM Z-Gly-Gly-Arg-AMC acetate [thrombin-specific fluorogenic substrate; MedChemExpress: HY-P0019A]) was added to the well(s), giving a final volume of 100 μL. An identical experiment was assembled in a separate plate without Z-Gly-Gly-Arg-AMC acetate, and with the addition of 200 nM rADAMTS13 mutants and 1 mg/mL GPRP-amide to prevent fibrin formation. The reaction was read for 90 minutes in 1-minute intervals on kinetic fluorescence mode (excitation=360 nm, emission=460 nm) using the SpectraMax M3 plate reader and the SoftMax Pro v7.1.2 software (Molecular Devices). Thrombin generation curves were generated using the Technothrombin® TGA Software (Technoclone, Vienna, Austria). To measure rADAMTS13 degradation, aliquots were removed at 0, 10, and 90 minutes after initiating thrombin generation. Samples were separated via SDS-PAGE under reducing conditions, and western blotting for the metalloprotease domain of ADAMTS13 was performed as previously described.

Proteolytic resistance of T4L/T8L-ADAMTS13 to fibrinolytic proteases in plasma: In a clear, flat-bottom 96-well plate, 3 μL of 1 M CaCl₂ and 5 μL of 1:5 tissue factor were added to achieve final concentrations of 30 mM and 1:100, respectively. In a separate tube, 50 μL of human platelet-poor pooled plasma, 0, 2, 6, or 10 nM activated tPA (Activase), 120 nM of wild type ADAMTS13 or T4L/T8L ADAMTS13, and HEPES-buffered saline (20 mM HEPES, 150 mM NaCl, pH 7.4) were combined. To examine rADAMTS13 and T4L/T8L degradation, an aliquot of this mixture was removed for the 0 timepoint. The mixture was then added to the plate to initiate the reaction at a final volume of 100 μL, and absorbance at 405 nm was read for 45 minutes using the SpectraMax M2 plate reader and SoftMax Pro v7.0.3 software. An aliquot was removed at 45 minutes for Western Blot analysis. Plasma samples were separated via SDS-PAGE under reducing conditions. Western blotting was performed for the metalloprotease domain of ADAMTS13 as previously described to visualize degradation.

Proteolytic resistance of T4L/T8L ADAMTS13 and T4L/T8L/1380G ADAMTS13 to activated neutrophils: To isolate neutrophils, 10 mL of citrated whole blood was combined with 2.5 mL of dextran (6% dextran in saline), then left to sediment the red blood cells for 45 minutes. The leukocyte-rich plasma layer was added to an equal volume of Histopaque 1077 (Sigma: 10771) and centrifuged at 240×g for 20 minutes. The supernatant was aspirated, and 2.5 mL of ACK lysis buffer (Gibco: A1049201) was added onto the pellet and incubated for 4 minutes. Hank's Balanced Salt Solution (Gibco: 14065-056) was then added to final volume of 50 mL, then the solution was centrifuged at approximately 240×g for 5 minutes, at 4° C., with maximum acceleration and deceleration. The supernatant was aspirated and the pellet was resuspended in 5 mL RPMI (Gibco: 11875093). The cells were counted using 0.4% trypan blue and a hemocytometer according to the manufacturer's protocol. The neutrophils were again pelleted, the supernatant was aspirated, and the pellet was resuspended at the desired concentration in RPMI.

For ADAMTS13 degradation assays, 0-500×10³ neutrophils diluted in RPMI were activated using 100 nM PMA for 4 hours at 37° C., at a volume of 10 μL. A reaction containing 100×10³ neutrophils without addition of PMA was also performed. 50 nM wild type ADAMTS13, T4L/T8L ADAMTS13, or T4L/T8L/1380G ADAMTS13 were added to the activated neutrophils to a final volume of 30 μL. The mixture was incubated for 1 hour at 37° C. Protein degradation after 1 hour was visualized via Western Blot.

### RESULTS AND DISCUSSION

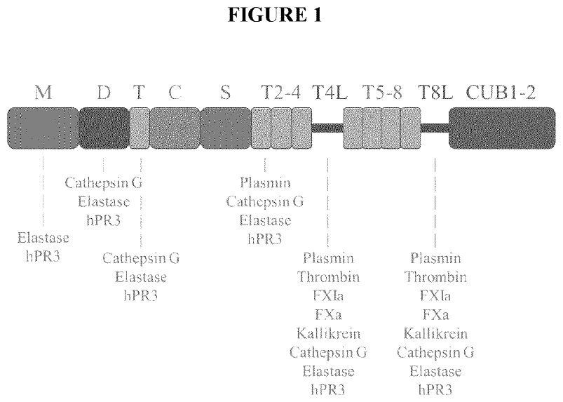

It is herein shown that ADAMTS13 is degraded by various proteases of the coagulation and fibrinolytic system and by proteases released by activated immune cells (such as neutrophils) (FIG. 1). These proteases (thrombin, plasmin, FXa, FXIa, kallikrein, cathepsin G, elastase, and HPR3) were found to result in a similar degradation pattern for ADAMTS13, suggesting similar cleavage sites.

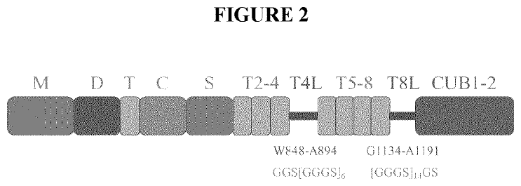

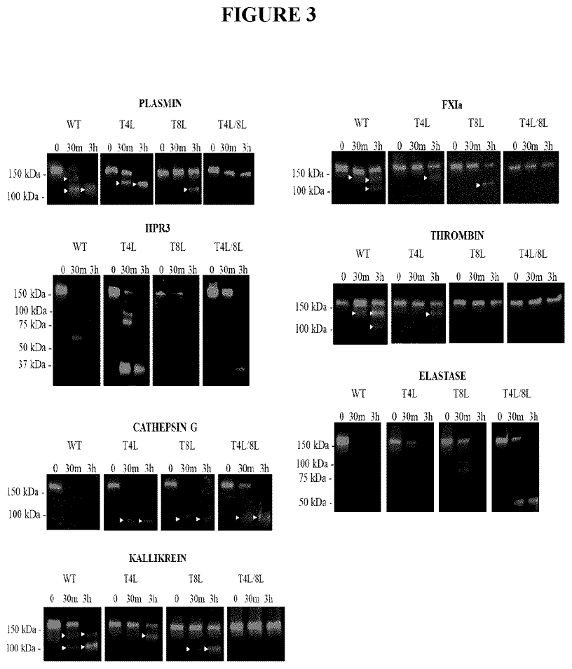

The cleavage sites were mapped to the linker regions connecting (a) TSP4 and TSP5 domains (T4-Linker) and (b) TSP8- and CUB1 domains (T8-Linker). These linker regions T4L (W848-A894) and T8L (G1134-A1191) were replaced with a glycine-serine hinge sequence GGS [GGGS]₆ and [GGGS]₁₄GS, respectively. We designed three variants of ADAMTS13 a) T4-Linker Mutant, b) T8-Linker Mutant, and c) T4&T8 Linker Mutant (FIG. 2). These mutant forms of ADAMTS13 were expressed in HEK293T cells and purified. We then compared their degradation to wild-type ADAMTS13 and discovered that the mutant forms of ADAMTS13 are not degraded by plasmin, kallikrein, FXa, thrombin, or FXIa (FIG. 3). The single mutant forms of ADAMTS13 are cleaved at the non-mutated site, whereas the double linker mutant is not cleaved at either location (FIG. 3). These mutants are partially resistant to neutrophil proteases (elastase, cathepsin G, and hPR3) (FIG. 3), suggesting that these proteases also target other regions within ADAMTS13.

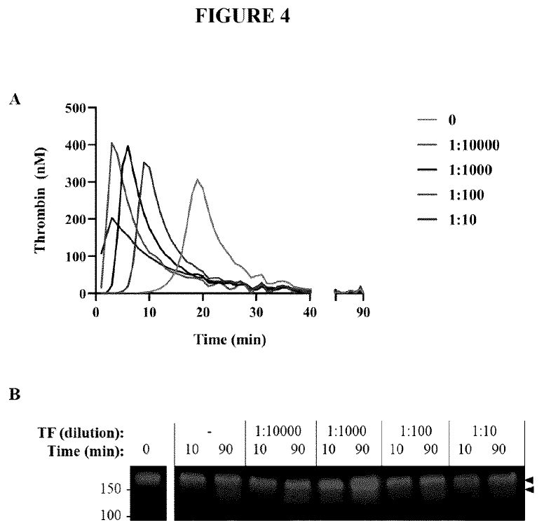

ADAMTS13 degradation in the plasma thrombin generation assay was determined. Results are shown in FIG. 4. Thrombin concentration was quantified over time by measuring fluorescence (ex=360 nm, em=460), and curves were generated using the Technothrombin TGA Software (FIG. 4A). Aliquots were removed prior to adding TF, and at 10 and 90 minutes after the addition of CaCl2 solution. Samples were separated via SDS-PAGE under reducing conditions, and ADAMTS13 degradation by thrombin was visualized via Western Blot using an anti-ADAMTS13 metalloprotease domain antibody (FIG. 4B). Molecular weight references are indicated on the left (kDa), and bands are indicated by black arrows.

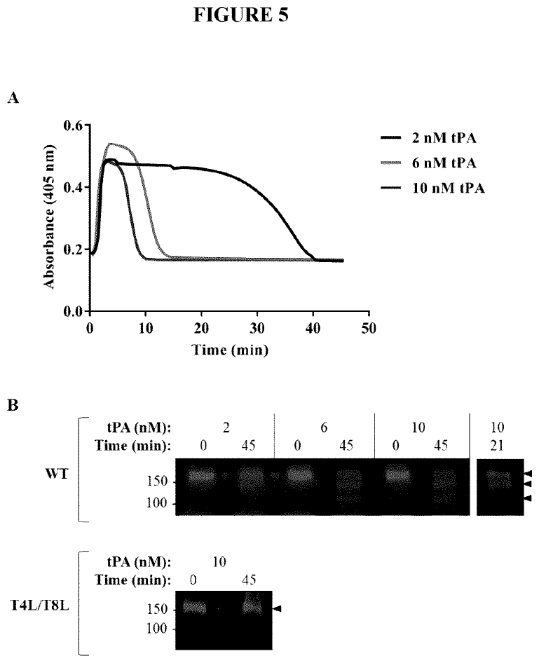

ADAMTS13 and T4L/T8L mutant degradation in the plasma fibrinolysis assay was determined. Plasma clot formation and clot lysis was quantified by measuring absorbance (405 nm) every 30 seconds. Absorbance increases with fibrin generation (FIG. 5A). Aliquots were removed at the indicated time points, and samples were separated via SDS-PAGE under reducing conditions. Cleavage was visualized via Western Blot using an anti-ADAMTS13 metalloprotease domain antibody. Molecular weight references are indicated on the left (kDa), and bands are indicated by black arrows. ADAMTS13 was cleaved by fibrin while the T4L/T8L mutant was not cleaved (FIG. 5B).

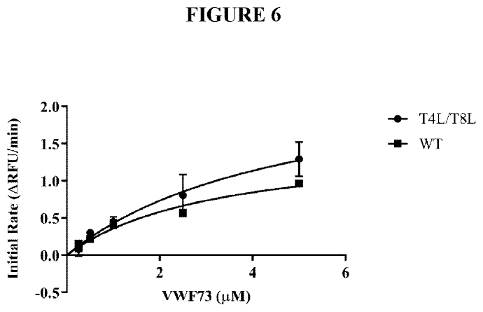

Michaelis-Menten analysis of ADAMTS13 and the T4L/T8L mutant VWF cleaving activity was measured using the FRETS-VWF73 assay. 20 nM WT or T4L/T8L was incubated with 0.25-5 μM FRETS-VWF73 substrate at 37° C. (n=3). Fluorescence was read every 15 seconds using a plate reader (ex=340 nm, em=450 nm). Linear regression was performed on initial data points (R2>0.8) to obtain initial reaction rates (ΔRFU/min), which were plotted against substrate concentration. As shown, the T4L/T8L mutant retains VWF cleaving activity (FIG. 6).

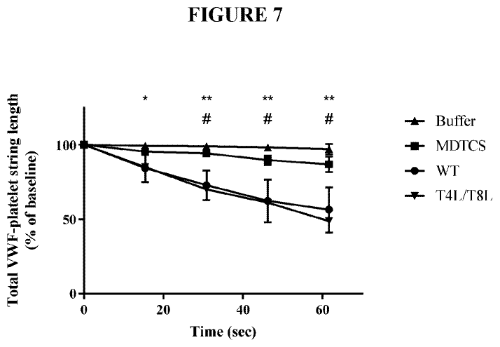

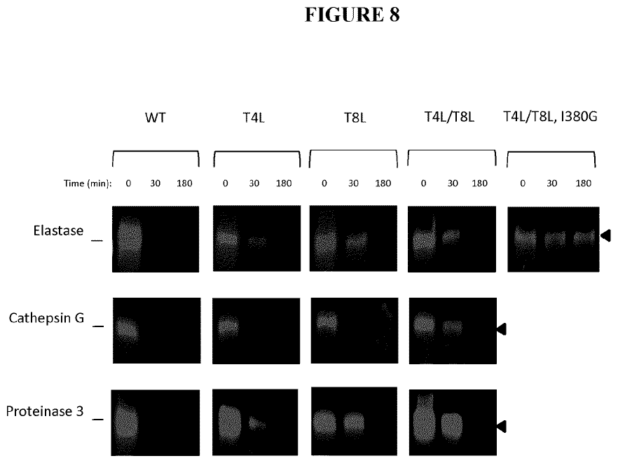

The VWF-cleaving activity of ADAMTS13, MDTCS (domain region of ADAMTS13), and the T4L/T8L mutant in a microfluidic flow assay was determined. DiOC6-stained platelets were perfused over a microfluidic flow chamber lined with HUVECs, shear-activating the cells and forming VWF-platelet strings. Channels were then perfused with 5 mL of either 5 nM WT, 5 nM MDTCS, 5 nM T4L/T8L mutant, or ADAMTS13 reaction buffer. Using a confocal microscope (10× dry lens; ex=490 nm, em=525 nm), videos were captured in a single frame at approximately the same channel region between experiments, at 0.77 frames/sec. The total length of strings within the frame was quantified every 20 frames for 5 frames, ending at the final frame captured before the channel dried out. Differences between groups were analyzed using a 2way ANOVA with multiple comparisons. *=WT vs. Buffer, p<0.05; **=WT vs. Buffer, p<0.01; #=WT vs. MDTCS, p<0.05. Both wild-type ADAMTS13 and T4L/T8L mutant exhibited antithrombolytic VWF-platelet string-reducing activity (FIG. 7).

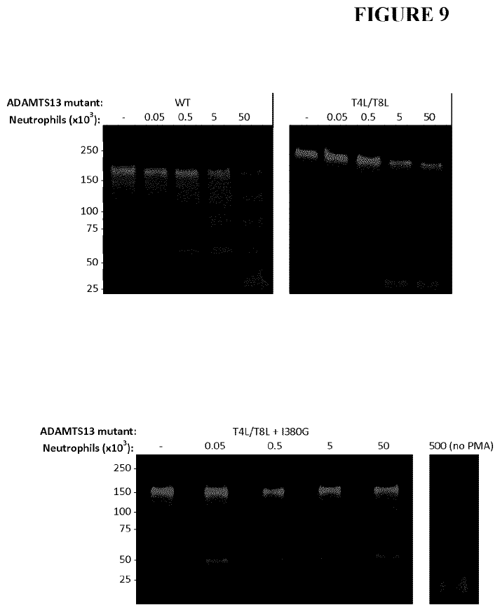

We next mapped the cleavage site for elastase to 1380 within the Distintegrin-like domain. We substituted this residue with a glycine (1380G) on top of the T4&T8 Linker Mutant to create a T4L/T8L/1380G Triple Mutant. This ADAMTS13 triple mutant was expressed in HEK293T cells and purified. We then compared the degradation to wild type ADAMTS13 and the T4/T8L double mutant and discovered that the triple mutant is additionally not cleaved by neutrophil elastase (FIG. 8). The Triple Mutant was also more resistant to degradation by activated neutrophils than T4L/T8L double mutant (FIG. 9)

While the present disclosure has been described with reference to examples, it is to be understood that the scope of the claims should not be limited by the embodiments outlined in the examples but should be given the broadest interpretation consistent with the description as a whole.

All publications, patents, and patent applications are incorporated by reference in their entirety to the same extent as if each individual publication, patent or patent application was specifically and individually indicated to be incorporated by reference. Where a term in the present application is found to be defined differently in a document incorporated herein by reference, the definition provided herein is to serve as the definition for the term.

### FULL CITATIONS FOR DOCUMENTS REFERRED TO IN THE APPLICATION

1. Crawley, J. T. B., de Groot, R., Xiang, Y., Luken, B. M., and Lane, D. A. (2011) Unraveling the scissile bond: How ADAMTS13 recognizes and cleaves von Willebrand factor. Blood. 10.1182/blood-2011-02-306597
2. Zhou, W., Inada, M., Lee, T. P., Benten, D., Lyubsky, S., Bouhassira, E. E., Gupta, S., and Tsai, H. M. (2005) ADAMTS13 is expressed in hepatic stellate cells. Laboratory Investigation. 10.1038/labinvest.3700275
3. Zheng, X., Majerus, E. M., and Sadler, J. E. (2002) ADAMTS13 and TTP. Current Opinion in Hematology. 10.1097/00062752-200209000-00001
4. Eerenberg, E. S., Teunissen, P. F. A., van den Born, B. J., Meijers, J. C. M., Hollander, M. R., Jansen, M., Tijssen, R., Beliën, J. A. M., van de Ven, P. M., Aly, M. F., Kamp, O., Niessen, H. W., Kamphuisen, P. W., Levi, M., and van Royen, N. (2016) The role of ADAMTS13 in acute myocardial infarction: Cause or consequence? Cardiovascular Research. 10.1093/cvr/cvw097
5. Chion, C. K. N. K., Doggen, C. J. M., Crawley, J. T. B., Lane, D. A., and Rosendaal, F. R. (2007) ADAMTS13 and von Willebrand factor and the risk of myocardial infarction in men. Blood. 10.1182/blood-2006-07-038166
6. Sonneveld, M. A. H., de Maat, M. P. M., Portegies, M. L. P., Kavousi, M., Hofman, A., Turecek, P. L., Rottensteiner, H., Scheiflinger, F., Koudstaal, P. J., Ikram, M. A., and Leebeek, F. W. G. (2015) Low ADAMTS13 activity is associated with an increased risk of ischemic stroke. Blood. 10.1182/blood-2015-05-643338
7. Chen, X., Cheng, X., Zhang, S., and Wu, D. (2019) ADAMTS13: An emerging target in stroke therapy. Frontiers in Neurology. 10.3389/fneur.2019.00772
8. Korkmaz, S., Keklik, M., Sivgin, S., Yildirim, R., Tombak, A., Kaya, M. E., Acik, D. Y., Esen, R., Hacioglu, S. K., Sencan, M., Kiki, I., Tiftik, E. N., Kuku, I., Okan, V., Yilmaz, M., Demir, C., Sari, I., Altuntas, F., Unal, A., and Ilhan, O. (2013) Therapeutic plasma exchange in patients with thrombotic thrombocytopenia purpura: A retrospective multicenter study. Transfusion and Apheresis Science. 10.1016/j.transci.2013.04.016
9. Wong, S. L., Goverman, J., Staudinger, C., and Wagner, D. D. (2020) Recombinant human ADAMTS13 treatment and anti-NET strategies enhance skin allograft survival in mice. American Journal of Transplantation. 10.1111/ajt.15703
10. Gandhi, C., Khan, M. M., Lentz, S. R., and Chauhan, A. K. (2012) ADAMTS13 reduces vascular inflammation and the development of early atherosclerosis in mice. Blood. 10.1182/blood-2011-09-376202
11. Garland, K. S., Reitsma, S. E., Shirai, T., Zilberman-Rudenko, J., Tucker, E. I., Gailani, D., Gruber, A., McCarty, O. J. T., and Puy, C. (2017) Removal of the C-terminal domains of ADAMTS13 by activated coagulation factor XI induces platelet adhesion on endothelial cells under flow conditions. Frontiers in Medicine. 10.3389/fmed.2017.00232
12. Spiel, A. O., Mayr, F. B., Ladani, N., Wagner, P. G., Schaub, R. G., Gilbert, J. C., and Jilma, B. (2009) The aptamer ARC1779 is a potent and specific inhibitor of von willebrand factor mediated ex vivo platelet function in acute myocardial infarction. Platelets. 10.1080/09537100903085927
13. Kageyama, S., Yamamoto, H., Nakazawa, H., Matsushita, J., Kouyama, T., Gonsho, A., Ikeda, Y., and Yoshimoto, R. (2002) Pharmacokinetics and pharmacodynamics of AJW200, a humanized monoclonal antibody to von Willebrand factor, in monkeys. Arteriosclerosis, Thrombosis, and Vascular Biology. 10.1161/hq0102.101520
14. Lei, X., Reheman, A., Hou, Y., Zhou, H., Wang, Y., Marshall, A. H., Liang, C., Dai, X., Li, B. X., Vanhoorelbeke, K., and Ni, H. (2013) Anfibatide, a novel GPIb complex antagonist, inhibits platelet adhesion and thrombus formation in vitro and in vivo in murine models of thrombosis. Thrombosis and Haemostasis. 10.1160/TH13-06-0490

1. A protease-resistant ADAMTS13 mutant protein or nucleic acid encoding the ADAMTS13 mutant protein, wherein the ADAMTS13 mutant protein comprises a mammalian ADAMTS13 protein in which one or more protease cleavage sites within the protein are replaced with amino acid sequence that is resistant to protease cleavage, and the mutant protein retains von Willebrand factor (VWF)-cleaving activity.

2. The protease-resistant ADAMTS13 mutant or nucleic acid encoding the ADAMTS13 mutant protein as defined in claim 1, wherein the ADAMTS13 protein is human protein or a functionally equivalent variant thereof.

3. The protease-resistant ADAMTS13 mutant or nucleic acid encoding the ADAMTS13 mutant protein as defined in claim 1 or claim 2, wherein the one or more protease cleavage sites comprise at least a thrombin sensitive cleavage site.

4. The protease-resistant ADAMTS13 mutant or nucleic acid encoding the ADAMTS13 mutant protein as defined in claim 1 or claim 2, wherein the mutant protein is resistant to cleavage by a protease involved in coagulation or fibrinolytic activity, or a protease released by activated neutrophils.

5. The protease-resistant ADAMTS13 mutant or nucleic acid encoding the ADAMTS13 mutant protein as defined in claim 4, wherein the protease is selected from the group of thrombin, plasmin, FXa, FXIa, kallikrein, cathepsin G, elastase, and HPR3.

6. The protease-resistant ADAMTS13 mutant or nucleic acid encoding the ADAMTS13 mutant protein as defined in any one of claims 1-4, wherein the protease cleavage site is selected from one or both of W848-A894 and G1134-A119 of the ADAMTS13 protein.

7. The protease-resistant ADAMTS13 mutant or nucleic acid encoding the ADAMTS13 mutant protein as defined in any one of claims 1-6, wherein the amino acid sequence that is resistant to protease cleavage is a glycine-rich sequence.

8. The protease-resistant ADAMTS13 mutant or nucleic acid encoding the ADAMTS13 mutant protein as defined in any one of claims 1-6, wherein the amino acid sequence that is resistant to protease cleavage comprises a GGS or GGGS repeat.

9. The protease-resistant ADAMTS13 mutant or nucleic acid encoding the ADAMTS13 mutant protein as defined in any one of claims 1-8, wherein the mutant additionally comprises a site-specific mutation within the disintegrin-like domain (Dis) which renders the mutant to be resistant to neutrophil elastase.

10. The protease-resistant ADAMTS13 mutant or nucleic acid encoding the ADAMTS13 mutant protein as defined in claim 9, wherein the Dis mutation is at amino acid position 380.

11. The protease-resistant ADAMTS13 mutant or nucleic acid encoding the ADAMTS13 mutant protein as defined in any one of claim 9 or 10, wherein the Dis mutation is I380G.

12. A composition comprising a protease-resistant ADAMTS13 mutant or nucleic acid encoding the ADAMTS13 mutant protein as defined in any one of claims 1-11 and a pharmaceutically acceptable carrier.

13. The composition of claim 12, additionally comprising a second therapeutic agent selected from an anticoagulant, a fibrinolytic, an antiplatelet, an anti-inflammatory and an analgesic agent.

14. A method of inhibiting or at least reducing thrombosis in a mammal comprising administering to the mammal a protease-resistant ADAMTS13 mutant protein or nucleic acid encoding the ADAMTS13 mutant protein as defined in any one of claims 1-11 or nucleic acid encoding the ADAMTS13 mutant protein.

15. The method of claim 14, to treat or prevent stroke, myocardial infarction, sepsis, venous thromboembolism, pulmonary embolism, microvascular thrombotic disorders, colitis, diabetes and atherosclerosis in the mammal.

16. The method of claim 14, wherein the mammal is treated with a dosage of about 0.1-500 mg per day of the protease-resistant ADAMTS13 mutant protein or an amount of nucleic acid that expresses about 0.1-500 mg of the ADAMTS13 mutant protein.

17. The method of any one of claims 14-16, wherein administration of the ADAMTS13 mutant protein or nucleic acid encoding the mutant protein results in a nonpathogenic hemostatic ADAMTS13/VWF axis.

18. The protease-resistant ADAMTS13 mutant or nucleic acid encoding the ADAMTS13 mutant protein as defined in any one of claims 1-11, the composition of any one of claims 12-13, or the method of any one of claims 14-17, comprising the sequence GGS[GGGS]₆ at amino acid position 848-894 or the sequence [GGGS]₁₄GS at position 1134-1191.

19. The protease-resistant ADAMTS13 mutant or nucleic acid encoding the ADAMTS13 mutant protein as defined in any one of claims 1-11, the composition of any one of claims 12-13, or the method of any one of claims 14-17, comprising the sequence GGS[GGGS]₆ at amino acid position 848-894 and the sequence [GGGS]₁₄GS at position 1134-1191.
